(19) United States
(12) Patent Application Publication
(10) Pub. No.: US 2023/0331778 A1
(43) Pub. Date: Oct. 19, 2023

(54) AN IMPROVED PROCESS FOR FMOC SYNTHESIS OF ETELCALCETIDE

(71) Applicant: USV Private Limited, Mumbai (IN)

(72) Inventors: Lester John Lobo, Mumbai (IN); Muralidharan Chandrakesan, Mumbai (IN); Chetan Doshi, Mumbai (IN); Shailesh Lalchand Chanadak, Mumbai (IN); Nandlal Gopal Yadav, Mumbai (IN); Nikhil Umesh Mohe, Mumbai (IN); Kodandaraman Vishwanathan, Mumbai (IN); Praful Shamrao Chavre, Mumbai (IN)

(21) Appl. No.: 18/021,391

(22) PCT Filed: Aug. 31, 2021
(86) PCT No.: PCT/IN2021/000006

§ 371 Date: Feb. 14, 2023

(30) Foreign Application Priority Data
Aug. 31, 2020 (IN) 202021037422

(51) Int. Cl. C07K 7/06; C07K 1/30; C07K 1/04

(52) U.S. Cl. CPC C07K 7/06; C07K 1/303; C07K 1/042

(57) ABSTRACT

The present invention relates to an improved process for the synthesis of Etelcalcetide and its analogs by solid phase synthesis of Fmoc protected amino acids in a sequential manner, followed by acetylation of terminal D-cys and cleavage of peptide from solid support. The crude heptapeptide thus obtained is reduced using Tris(2-carboxyethyl) phosphine hydrochloride, purified and oxidized with L-cysteine. The oxidized Etelcalcetide is purified and salt exchanged using a one-step reverse phase chromatography process. The purified Etelcalcetide hydrochloride is then precipitated using organic solvents, concentrated and lyophilized to purity of greater than 99.0%.

### RELATED APPLICATION

This application claims benefits of Indian Provisional Application No. 202021037422 filed on 31st Aug. 2020

### FIELD OF THE INVENTION

The present invention relates to an improved process for the synthesis of Etelcalcetide hydrochloride having an amino acid sequence as set forth in Formula I.

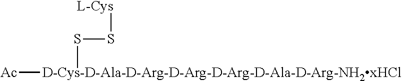

### BACKGROUND OF THE INVENTION

Etelcalcetide is a synthetic peptide calcimimetic agonist calcium-sensing receptor (CaSR) developed for the treatment of secondary hyperparathyroidism (sHPT) in patients with chronic kidney disease (CKD). The hydrochloride salt of Etelcalcetide is described chemically as N-acetyl-D-cysteinyl-S-(L-cysteine disulfide)-D-alanyl-D-arginyl-D-arginyl-D-arginyl-D-alanyl-D-argininamide hydrochloride.

Hyperparathyroidism is the condition of elevated parathyroid hormone (PTH) levels and is often observed in people with chronic kidney disease. Chronic kidney failure is the most common cause of secondary hyperparathyroidism. Failing kidneys do not convert enough vitamin D to its active form, and they do not adequately excrete phosphate. When this happens, insoluble calcium phosphate forms in the body and removes calcium from the circulation. This leads to hypocalcemia and a subsequent increase in parathyroid hormone secretion in an attempt to increase the serum calcium levels. Both processes lead to hypocalcemia and hence secondary hyperparathyroidism.

The secondary hyperparathyroidism is a major complication in patients with chronic kidney disease. sHPT is related to chronic kidney disease-mineral bone disorder, leading to increased morbidity and mortality. Etelcalcetide is intravenously administered at the end of hemodialysis (HD).

Etelcalcetide is a calcimimetic agent that allosterically modulates the calcium-sensing receptor. Etelcalcetide binds to the CaSR and enhances activation of the receptor by extracellular calcium. Activation of the CaSR on parathyroid chief cells decreases PTH secretion. Etelcalcetide is a synthetically produced white to off-white powder with a molecular formula of C₃₈H₇₃N₂₁O₁₀S₂⋅xHCl (4≤x≤5).

Various processes of peptide synthesis are known in the prior arts. Both solution and solid phase synthesis are used in peptide synthesis. Solution phase of peptide synthesis uses homogeneous phase during reaction whereas solid phase peptide synthesis uses a solid support such as resin at the beginning of the reaction. First a. protected amino acid binds to such solid support. Subsequent protected amino acids are added to the resin bound amino acid chain to get the desired peptide sequence. Once the sequence of interest is synthesized on solid support, peptide is cleaved from solid support followed by isolation and purification to the desired purity.

Amino acids have reactive side-chains in addition to the reactive end terminals. Thus in order to get the desired sequence, unwanted reactions occurring at the side chain needs to be controlled. Uncontrolled side reaction leads to multiple undesired byproducts which affect the quality and yield of the end product. To minimize side-chain reactions, it is conventional practice to appropriately protect the reactive side-chains of the amino acid to ensure that desired reaction occurs at the end terminal of the amino acid.

Etelcalcetide is a 1047.5 Dalton synthetic octapeptide which consists of seven D-amino acid heptamer linked to L-cysteine via a disulfide bond. General practice is to synthesize 7 amino acid linear chain by peptide synthesis. C-terminal is amidated and N-terminal is acetylated. D-cysteine at the N-terminal is attached to the L-cysteine through disulfide bond. Synthesis of Etelcalcetide is challenging due to presence of seven D amino acids in its structure and the attachment of L-cysteine to the linear chain of 7 amino acids also leads to the formation of impurities. Disulfide bond formation between two heptapeptide residues leads to formation of dimer Etelcalcetide. Presence of dimer impurity leads to reduced yield and quality of drug substance. Controlling impurities in the drug substance is a crucial regulatory requirement. Hence robust manufacturing process having optimum yields and better productivity is the need of the hour.

Various approaches are used in the patent applications to ensure correct formation of disulfide bond.

WO2016154580 describes use of solution phase synthesis of Etelcalcetide. In this method Etelcalcetide is synthesized by, using primary amino acids D-ornithine , instead of D-arginine. The heptapeptide is synthesized by merging two different size fragments together. After synthesizing the heptapeptide, oxidative cleavage is carried out using iodine followed by coupling with L-cysteine to get Etelcalcetide. 2-pyridinesulfenyl (Spy/S-Pyr) is used as protecting group of D-cysteine which will help selective reaction of D-cysteine and L-cysteine producing Etelcalcetide with good yield and purity. However, solution phase synthesis is time consuming and a laborious process especially during scale up to higher volumes. Further, testing the quality of the isolated intermediate is a prerequisite in solution phase synthesis, which further increases the cycle time of a solution phase synthesized peptide. Thus there is a need to reduce cycle time of the peptide synthesis. Ease of scaling up of process is another important factor for consideration of industrial production of peptides. Various solid phase peptide syntheses which shorten synthesis cycle time, are used in various prior arts.

US2019100554 uses Fmoc approach for peptide synthesis followed by deprotection. Post acetylation, protecting group 3-nitro 2-pyridine-sulfenyl (Npys) is used to protect side chains of L-cysteine, which ensures correct disulfide bond formation. Cleavage of the peptide is carried out using mixture of triisopropylsilane (TIPS), thioanisole, trifluroacetate (TFA) and water.

WO2017114238 is based on use of N-Chlorosuccinimide which carries out chlorination of L-cysteine. The hydrogen on the sulfhydryl group is replaced by chlorine to form L-Cys (SC1) which is then coupled with linear heptapeptide of D amino acids to form Etelcalcetide. Coupling reaction is carried out using mixture of dichloromethane (DCM) and trifluroacetic acid.

CN105504012 describes linear synthesis of D amino acid heptapeptide which involves the use of mtt/mmt group on D cysteine which is easily removed using a 5% TFA in MDC solution. The attachment of the free Cysteine is facilitated by using D-Cys(Npys) based protected amino, acid. The crude purity obtained from the above process is nearly 64%. The use of a base like DIPEA for the coupling of the X-Cys (npys) to the linear peptide may induce the formation of racemic impurities which are difficult to separate on a reverse phase purification process. Further, the final process yield is ˜30%, which is low for a octamer peptide generated by solid phase peptide synthesis.

CN107434820 uses Trt or Mmt or StBu, as protecting group for D-cysteine to assure disulfide bond formation with the L-cysteine. The octapeptide so generated is cleaved using mixture of reagents of TFA/PhSMe/EDT/TIS/H2O. Various prior arts cited above outlines use of specific protecting groups or fragment approach to ensure correct disulfide bond formation which in turn leads to reduction in overall impurities in the final end product. The specially protected amino acid are quite expensive which increases overall cost of the process during large-scale production. Use of regular amino acids without any specific protecting groups leads to incorrect formation of disulfide bond which in turn leads to high amount of impurities in the end product. A cost-effective process with use of mild reaction conditions yielding highly purified Etelcalcetide peptide is the gap required to be filled. The process of the present invention is primarily a solid phase synthesis which uses a solution phase reaction for the attachment of free cysteine to the heptamer of Etelcalcetide. The coupling and cleavage process ensures that a crude purity of greater than 90% is achieved. Further a HPLC purification ensures the removal of all impurities from the heptamer peptide prior to its oxidation.

The purified heptamer peptide, with purity above 98% is oxidized using a combination of a low cost and readily available free L-cysteine. A green chemistry oxidation catalyst namely hydrogen peroxide is used to make the process eco-friendly. The final process yield for the process of the present invention is 45 to 50% with a final purity of greater than 99.8%. Further the process of the present invention has a high throughput and is cost effective in nature. Another advantage is that it uses regular commercially available and relatively lower cost protected amino acids making the process commercially feasible.

### OBJECT OF THE INVENTION

An object of the present invention is to provide a simple, cost-effective, reproducible, commercially viable and industrially feasible process for preparation of Etelcalcetide.

Another object of the present invention is to provide substantially pure Etelcalcetide.

### SUMMARY OF THE INVENTION

According to one aspect of the present invention, there is provided an improved process for the synthesis of Etelcalcetide hydrochloride using FMOC based solid phase synthesis comprising the steps of
a) synthesizing crude heptamer-etelcalcetide of Fmoc protected amino acids in a sequential manner, followed by acetylation of terminal D-cys,
b) cleavage of peptide from solid support using combination of TFA with scavengers TIS and DMS and reducing agent TCEP,
c) optionally purifying crude heptamer-etelcalcetide and oxidizing it with free L-cysteine to Etelcalcetide,
d) purifying Etelcalcetide using a one-step reverse phase chromatography process to Etelcalcetide hydrochloride to a purity of at least 99.8%,
e) precipitating, adjusting chloride salt content and lyophilizing the concentrated purified peptide solution.

Preferably, purification of crude heptamer-etelcalcetide is carried out by gradient mode of RP-HPLC comprising of perchloric acid buffer system with pH in the range of 3 to 5, preferably 2.5 as an aqueous phase and acetonitrile as an organic phase with isolated yield of at least 50% and purity of at least 99%.

Preferably, one step purification of Etelcalcetide is carried out by gradient mode of RP-HPLC comprising of Ammonium chloride in Hydrochloric acid buffer system as an aqueous phase with pH in the range of 2 to 5, preferably 3 and an organic phase comprising from solvents selected from methanol, ethanol or acetonitrile preferably methanol; to Etelcalcetide hydrochloride with isolated yield of at least 43% and purity of at least 99.8%.

Preferably, the precipitation of purified Etelcalcetide hydrochloride is by solvent system comprising of mixture of solvents consisting from group of methanol, ethanol, isopropyl alcohol, acetonitrile, ethyl acetate, acetone either alone or in combination thereof, most preferably combination of ethanol and acetone.

Preferably, washing the precipitate of purified Etelcalcetide hydrochloride with an acidified solution of acetone or a mixture of acetone and ethanol, which consists of 0.05% to 2% hydrochloric acid in acetone, or mixture of acetone: ethanol, most preferably 0.25% hydrochloric acid in acetone to ensure the counter ion content of the Etelcalcetide is maintained within a narrow range of 4 to 5 equivalents with respect to the peptide.

Preferably, the adjustment of chloride salt content involves addition of chilled solution of dil. Hydrochloric acid in water to the peptide, the concentration of which is maintained at concentration of 50 to 200 mg/ml more preferably 200 mg/ml.

Preferably, the lyophilization of the purified Etelcalcetide hydrochloride is done at the high peptide concentration ranging from 50 to 300 mg/ml, more preferably at 200 mg/ml to obtain an amorphous peptide with a high bulk density of 0.6-1.0 g/cm³.

### BRIEF DESCRIPTION OF THE ACCOMPANYING DRAWINGS

FIG. 1 shows analytical RP-HPLC profile on 4.6 mm×250 mm C18, 2.6 μ column of chemically synthesized crude heptamer Etelcalcetide after cleavage by the process of EXAMPLE 2A of the present invention.

FIG. 2 shows analytical RP-HPLC profile on 4.6 mm×250 mm C18, 2.6 μ column of chemically synthesized crude heptamer Etelcalcetide after cleavage by the process of EXAMPLE 2B of the present invention

FIG. 3 shows analytical RP-HPLC profile on 4.6 mm×250 mm C18, 2.6 μ column of chemically synthesized crude heptamer Etelcalcetide obtained after reduction by the process of EXAMPLE 3 of the present invention

FIG. 4 shows analytical RP-HPLC profile on 4.6 mm×250 mm C18, 2.6 μ column of chemically synthesized heptamer Etelcalcetide obtained after purification by the process of EXAMPLE 4 of the present invention

FIG. 5 shows analytical RP-HPLC profile on 4.6 mm×250 mm C18, 2.6 μ column of chemically synthesized Etelcalcetide obtained after oxidation by the process of EXAMPLE 5 of the present invention

FIG. 6 shows analytical RP-HPLC profile on 4.6 mm×250 mm C18, 2.6 μ column of chemically synthesized purified Etelcalcetide obtained after purification by the process of EXAMPLE 5 of the present invention

FIG. 7 shows analytical RP-HPLC profile on 4.6 mm×250 mm C18, 2.6 μ column of chemically synthesized purified Etelcalcetide obtained after purification by the process of EXAMPLE 6 of the present invention

### DETAILED DESCRIPTION OF THE INVENTION:

In a preferred embodiment, the present invention provides an improved process for synthesis of Etelcalcetide or salt or precursor thereof as set forth in Formula I by an orthogonal Fmoc strategy comprising of:

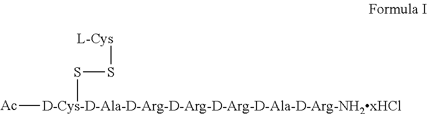

i. covalently linking a Fmoc-D-Arg(Pbf)-OH to polystyrene based solid resin support,
ii. removing the α-NH2 protecting group from Fmoc-D-Arg(Pbf)-solid support to obtain a free α-NH2 group,
iii. coupling the second Fmoc-D-Ala to the D-Arg(Pbf)-solid support, by activating the amino acid by DIC/HOBT in the presence of organic solvent,
iv. deprotecting the Fmoc group by a deprotectant,
v. repeating steps ii), iii), iv) for assembling the heptapeptide H-D-Cys(Trt)-D-Ala-D-Arg(Pbf)-D-Arg(Pbf)-D-Arg(Pbf)-D-Ala-D-Arg(Pbf)-solid resin support,
vi. acetylating the N-terminal group to produce acetylated heptapeptide Ac-D-Cys(Trt)-D-Ala-D-Arg(Pbf)-D-Arg(Pbf)-D-Arg(Pbf)-D-Ala-D-Arg(Pbf)-solid resin support,
vii. cleaving the heptapeptide from solid resin support using cleavage cocktail consisting of TFA in the range of 80 to 95% V/V, TIS in the range of 2 to 10% V/V, DMS in the range of 2 to 10% V/V, TCEP in the range of 1 to 5% V/V, water in the range of 1 to 5% V/V, preferably TFA: TIS: DMS: TCEP: water in the ratio of 85: 6.5: 3.5:1.5:1 (%v/v); to obtain crude heptamer-etelcalcetide as set in the formula II of at least 90% peptide purity,

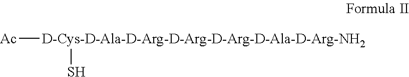

viii. optionally purifying the crude heptamer-etelcalcetide as set in the formula II of step vii) by chromatography wherein said peptide has a purity of ≥98%,
ix. oxidizing heptamer-etelcalcetide as set in the formula II of step viii) with free single cysteine in presence of hydrogen peroxide to obtain Etelcalcetide as set forth formula I
x. purifying Etelcalcetide hydrochloride of step ix) by reverse phase HPLC to a purity of at least 99.8%
xi. precipitating, adjusting chloride salt content and lyophilizing the concentrated purified peptide solution of step x).

In another preferred embodiment, the present invention provides an improved process for synthesis of Etelcalcetide or salt/precursor thereof as set forth in Formula 1 by an orthogonal Fmoc strategy comprising of:
i. covalently linking a Fmoc-D-Arg(Pbf)-OH to polystyrene based solid support,
ii. removing the α-NH2 protccting group from Fmoc-D-Arg(Pbf)-solid resin support to obtain a free α-NH2 group,
iii. coupling the Fmoc-D-Ala to the D-Arg(Pbf)-solid resin support, by activating the amino acid by DIC/HOBT in the presence of organic solvent,
iv. deprotecting the Fmoc group by deprotectant,
v. repeating steps ii), iii), iv) for assembling the heptapeptide H-D-Cys(Trt)-D-Ala-D-Arg(Pbf)-D-Arg(Pbf)-D-Arg(Pbf)-D-Ala-D-Arg(Pbf)-solid resin support,
vi. acetylating the N-terminal group to produce acetylated heptapeptide Ac-D-Cys(Trt)-D-Ala-D-Arg(Pbf)-D-Arg(Pbf)-D-Arg(Pbf)-D-Ala-D-Arg(Pbf)-solid resin support,
vii. cleaving the heptapeptide from solid resin support using cleavage cocktail consisting of TFA in the range of 80 to 95% V/V, TIS in the range of 2.5 to 10% V/V, DMS in the range of 2.5 to 10% V/V, preferably TFA: TIS: DMS in the ratio of 90: 6.5: 3.5 (%v/v) to obtain mixture of crude heptamer-etelcalcetide as set in the formula II and dimer-etelcalcetide as set in the formula III,

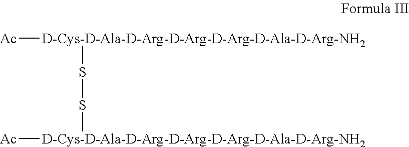

viii. pretreating the crude mixture of heptamer-etelcalcetide and dimer-etelcalcetide of step vii) with Tris(2-carboxyethyl)phosphine hydrochloride in presence of perchloric acid buffer having concentration ranging from 0.1 to 2% preferably 1% and pH ranging from 2 to 5, preferably 2.5 to obtain heptamer-etelcalcetide of at least 90% peptide purity,
ix. purifying the crude heptamer-etelcalcetide as set in the formula II of step viii) by chromatography wherein said peptide has a purity of ≥98%,
x. oxidizing heptamer-etelcalcetide as set in the formula II of step ix) with free single cysteine in presence of Hydrogen peroxide to obtain Etelcalcetide as set forth formula I
xi. purifying Etelcalcetide hydrochloride of step x) by reverse phase HPLC to a purity of at least 99.8%
xii. precipitating, adjusting chloride salt content and lyophilizing the concentrated purified peptide solution of step xi).

In preferred embodiment of the invention the process uses TCEP as reducing agent in cleavage cocktail or pre treatment of cleaved peptide with TCEP. Cleavage of heptamer Etelcalcetide from the peptidyl resin leads to the formation of significant levels of dimer Etelcalcetide by the formation of the disulfide bridge between two units of heptamer Etelcalcetide molecules. This reaction can be attributed to the inductive effect of the neighboring amino acids to D-cysteine. The use of TCEP in the cleavage cocktail prevents the formation of the dimer Etelcalcetide and. ence the crude peptide generated is principally composed of only heptamer Etelcalcetide.

Alternatively the dimer Etelcalcetide in the crude peptide generated without the use of TCEP in the cocktail can be converted completely to heptamer Etelcalcetide by the use of TECP prior to the purification of HPLC. The sample preparation involves the reduction of the dimer Etelcalcetide to heptamer Etelcalcetide and its subsequent purification. As TCEP is a cost effective reducing agent which works in the acidic pH range its use in the cleavage cocktail or in pre purification process is commercially viable and helps generate crude peptide with higher purity.

Another embodiment of the present invention provides a process wherein the organic solvent used in the coupling is selected from a group consisting of DMF, NMP, mixture of DMF and MDC or any combination thereof, more preferably DMF.

Still another embodiment of the present invention provides a process wherein cleavage of heptapeptide from the solid resin support is carried out using mixture of solvents selected from a group consisting of TFA, TCEP, TIS, Water, DTT, DMS, DODT.

Yet another embodiment of the present invention provides a process wherein cleavage of heptapeptide from the solid resin support is done wherein concentration of peptidyl resin is 10-30 ml/g, preferably 20 ml/g.

In yet another embodiment of the present invention provides a process wherein the purification of crude heptamer-etelcalcetide to a purity of ≥99% carried by chromatography is by RP-HPLC using isocratic and/or gradient mode.

In another embodiment of the present invention provides the process wherein the eluent for RP-HPLC purification of crude heptamer-etelcalcetide by gradient mode comprises of perchloric acid buffer system with pH in the range of 3 to 5, preferably 2.5 as an aqueous phase and acetonitrile as an organic phase with isolated yield of at least 50% and purity of at least 99%.

In another embodiment of the present invention provides the process for purification of Etelcalcetide which is a highly hydrophilic peptide owing to the presence of high content of Arginine residues. The hydrophilicity poses challenges to bind the peptide to a reverse phase column for its purification. The use of Perchloric acid buffer system helps to bind such hydrophilic peptides to reverse phase HPLC column which is mediated through the salt formation of perchlorate ion with the guanido nitrogen of Arginine residues in the peptide thus enabling their purification. Further, Perchloric acid is more economically viable at commercial scales as compared to strong ion pairing agents like Alkane Sulphonic acids, which are also difficult to exchange from the peptide during salt exchange step affecting complete counter ion formation.

In still another embodiment of the present invention provides the process wherein the purification of Etelcalcetide and its conversion to its hydrochloride salt with purity of ≥99.80% by chromatography is by RP-HPLC using isocratic and/or gradient mode.

In yet another embodiment of the present invention provides the process wherein the eluent for RP-HPLC purification of Etelcalcetide by gradient mode comprises of Ammonium chloride in Hydrochloric acid buffer system as an aqueous phase with pH in the range of 2 to 5, preferably 3 and an organic phase comprising from solvents selected from methanol, ethanol or acetonitrile preferably methanol with isolated yield of at least 43% and purity of at least 99.8%.

In yet another embodiment of the present invention provides the process wherein the use of Ammonium chloride as a buffer system enables the chloride salt formation of the peptide and also subsequent removal of the perchlorate counter ion from the peptide. The pH of the buffer is maintained at a range wherein aqueous solutions of Etelcalcetide are highly stable. Ammonium chloride buffer is also soluble in ethanol which facilitates its easy removal during the peptide precipitation step.

Another embodiment of the present invention provides the process wherein the precipitation of purified Etelcalcetide hydrochloride is by solvent system comprising of mixture of solvents consisting from group of methanol, ethanol, isopropyl alcohol, acetonitrile, ethyl acetate, acetone either alone or in combination thereof, most preferably combination of ethanol and acetone.

Another embodiment of the present invention provides the process wherein the precipitation of Etelcalcetide hydrochloride using a solvent system ensures the removal of excess buffer components from the peptide simultaneously while isolating the peptide. This step offers a more economically and easily scalable process as compared to nanofiltration process involving buffer exchange.

Another embodiment of the present invention provides the process wherein the predipitation of purified Etelcalcetide hydrochloride is followed by washing the precipitate obtained with an acidified solution of acetone or a mixture of acetone and ethanol, which consists of 0.05% to 2% hydrochloric acid in acetone, or mixture of acetone: ethanol, most preferably 0.25% hydrochloric acid in acetone to ensure the counter ion content of the Etelcalcetide is maintained within a narrow range of 4 to 5 equivalents with respect to the peptide.

Another embodiment of the present invention provides the process wherein washing of the wet cake of precipitated peptide using acidified acetone allows for the formation of the precise stoichiometric content of the chloride counter ion.

In yet another embodiment of the present invention provides the process wherein the adjustment of chloride salt content involves addition of chilled solution of dil. HCl in water to the peptide, the concentration of which is maintained at concentration of 50 to 200 mg/ml more preferably 200 mg/ml.

In yet another embodiment of the present invention provides the process wherein high concentration lyophilization ensures a high bulk density and low surface area of the resulting lyophilised peptide. This helps to prevent significant absorption of moisture, ensuring better long term stability.

In other embodiment of the present invention provides the process wherein the lyophilization of the purified Etelcalcetide hydrochloride is done at the high peptide concentration ranging from 50 to 300 mg/ml, more preferably at 200 mg/ml to obtain an amorphous peptide with a high bulk density of 0.6-1.0 g /cm³.

In other embodiment of the present invention provides the process wherein the chloride content of the peptide significantly impacts the stability of Etelcalcetide resulting in the formation of acid Etelcalcetide or dimer Etelcalcetide impurities. Adjustment of the chloride content prior to lyophilization ensures the precise stoichiometric ratio of the chloride counterion of 4 to 5 with respect to Etelcalcetide is maintained.

In yet another embodiment of the present invention provides the process of purification of Etelcalcetide hydrochloride with the overall yield of at least 43% and a purity of at least 99.8%.

Drawings accompanying the specification gives the analytical purity obtained post purification by RP-HPLC at intermittent steps. The foregoing descriptions are merely preferred embodiments of the present invention and are not intended to limit the present invention. For those skilled in the art, the present invention may have various changes and modifications. Any modification, equivalent replacement, Sand improvement made within the spirit and principle of the present invention shall fall within the protection scope of the present invention.

### EXAMPLES

The present invention is described in more detail below with reference to some illustrative examples. It should be understood, however, that these examples serve only to facilitate understanding of the invention but not to restrict the scope of the invention. Unless otherwise specified, the reagents and instruments used in the examples are common commercially available products.

Glossary of terms used in the specification:
AA: Amino acid
CaSR: calcium-sensing receptor
CKD: chronic kidney disease
DBU: 1,8-Diazabicycloundec-7-ene
DCM: dichloromethane
DIC: N, N Di-isopropylcarbodiimide
DIPE: Diisopropyl ether
DIPEA: N,N Di-sisopropyl ethylamine
DMF: Dimethyl formamide
DMS: Dimethyl sulfide
DTT: Dithiothreitol
DODT: 2,2′-(Ethylenedioxy)diethanethiol
EDT: EthaneDithiol
Eq: Equivalent
HBTU: Hydroxybenzotriazole Uronium Salt
HD: Hemodialysis
HoBT: Hydroxybenzotriazole monohydrate
HPLC: High performance liquid chromatography
MBHA: Methylbenzyhydrylamine
MDC: Methylene Dichloride
mmt: Monomethoxytrityl
MTBE: Methyl tertiary butyl ether
NMM: N-methyl morpholine
NMP: N-Methyl pyrrolidone
Npys: 3-nitro-2-pyridinesulfenyl
pbf:2,2,4,6,7-Pentamethyldihydrobenzofuran-5-sulfonyl
PhSMe: Thioanisole
PTH: Parathyroid hormone
RP-HPLC: Reverse phase high performance liquid chromatography
RT: Room temperature
sHPT: secondary hyperparathyroidism
Spy/S-Pyr: 2-pyridinesulfenyl
StBu: S-tertiary butyl
TCEP: Tris(2-carboxyethyl)phosphine hydrochloride
TIS/TIPS: Tri isopropyl silane
TFA: Trifluoroacetic acid
trt: trityl

As used herein ther term “D-amino acid” refers to dextro isomer of an amino acid

As used herein ther term “heptamer Etelcalcetide” refers to 7 D-amino acid sequence as specified in formula II

As used herein ther term “dimer Etelcalcetide” refers to sequence of 14 D-amino acid as specified in formula III formed due to disulfide bond formation between two heptamer Etelcalcetide

As used herein ther term “deprotectant” refers to any reagent used for removing the N-α-amino protecting group in the present invention reference is herby made to Fmoc.

As used herein ther term “orthogonal Fmoc strategy” refers to an approach which uses the base-labile N-Fmoc group for protection of the α-amino function, and acid labile side chain protecting groups.

### Chemical Synthesis of Etelcalcetide Hydrochloride Using FMOC Based Solid Phase Peptide Synthesis

### EXAMPLE 1: Synthesis of the Heptamer on the Solid Support SPPS Synthesis of Acetyl-D-Cys(Trt)-D-Ala-D-Arg(Pbf)-D-Arg(Pbf)-D-Arg(Pbf)-D-Ala-D-Arg(Pbf)-Rink Amide Resin

The peptide was synthesized as the peptide amide by the solid phase peptide synthesis technology on the Rink Amide AM resin using Fmoc chemistry.

| | |
|---|---|
| Instrument | Automated peptide synthesiser |
| Solid Support | Fmoc Rink amide AM resin |
| Excess Ratio of Reagents | Amino Acid (3.5 Eq):DIC (3.5 Eq): |
| | HOBT (3.5 Eq) |
| Coupling solvent | DMF |
| Coupling steps | Coupling for 60 mins, 6 washes with DMF |
| | for 2 minutes each |
| Deprotection solution | 20% piperidine in DMF |
| Deprotection step | Deprotection for 2 and 10 minutes, 6 washes |
| | with DMF for 2 minutes each |
| Capping Solution | Acetic Anhydride (5 Eq), NMM (7.5 Eq) |

Fmoc-Rink Amide AM Resin 50.77 g (33 mmol) was swelled in 355 ml DMF for 30 min, drained and deblocked with 355 ml of 20% Piperidine solution in DMF for 2 minutes and 10 minutes respectively. Alternatively, 2% DBU in DMF or Mixture of both 20% Piperidine solution in DMF and 2% DBU in DMF can be used for deblocking. Deblocking of the Fmoc Rink amide AM resin was followed by washings with DMF 350 ml for 6 times. Polystyrene based supports used are selected from group of Rink Amide AM Resin, Rink Amide Resin, Rink Amide MBHA Resin more preferably Rink Amide AM Resin. Loading of Fmoc Rink Amide AM Resin selected was from 0.28-1.12 mmole/g, preferably 0.2 to 0.55 mmol/g, more preferably 0.4 to 0.5 mmole/g.

### Step I: Coupling of Fmoc-D-Arg(Pbf)-OH to Rink Amide AM Resin

33.1 g of Fmoc-D-Arg(Pbf)-OH (1.5 eq, 5.1 mmol) and 7.6 g of HOBt.H₂O (1.5 eq, 4.96 mmol) was dissolved in 350 ml DMF and transferred in to deblocked Rink amide AM resin. This was followed by dropwise addition of 7.8 ml of DIC (1.5 eq) and stirred for 2 hrs. After coupling, peptidyl resin washed with DMF (350 ml) for 2 min each for 5 cycles. Acetic anhydride (15.9 ml, 5 eq, 16.85 mmol) and NMM (27.1 ml, 7.5 eq, 24.64 mmol) in 350 ml DMF is used for 30 min to carry out capping. The purpose of this step is to cap unreacted amines on rink amide resin so that the next amino acids coupled are not attached to the resin. After capping peptidyl resin is washed with DMF (350 ml) for 2 min each for 5 cycles and again washed with Dichloromethane(350 ml) for 2 min each for 3 cycles and dried under vacuum. Alternative solvent systems used for carrying out this attachment was uronium based reagent in presence of base lke NMM or DIPEA or mixture of NMM and DIPEA in solvents like DMF, MDC or mixtures thereof.
Weight of Fmoc-D-Arg(Pbf)-Rink Amide AM Resin: 61.4 g
Yield: 84%
   Step II: Synthesis of Acetyl-D-Cys(Trt)-D-Ala-D-Arg(Pbf)-D-Arg(Pbf)-D-Arg(Pbf)-D-Ala-D-Arg(Pbf)-Rink Amide AM Resin from Fmoc-D-Arg(Pbf)-Rink Amide AM Resin

The couplings of the remaining amino acids were carried out ,in a similar way, by , repeating the above cycle, till the desired sequence length was attained. The assembly of the peptide chain is carried out in the following manner.

The Fmoc-D-Arg(Pbf)-Rink Amide AM Resin was weighed on the basis of the scale of the synthesis. 45.41 grams(20 mmol) of Fmoc-D-Arg(Pbf)-Rink Amide AM resin was swollen in DMF. The synthesis of the linear chain on the solid support involves the sequential deblocking of the Fmoc group of the attached amino acid and the activation and coupling of the protected amino acid. The coupling is established using DIC/HOBT in the ratio of AA: DIC: HOBT in 3.5 equivalents each. The reaction was carried out for 1 Hr in organic solvents selected from a group consisting of DMF, NMP, mixture of DMF and MDC or combination of stated solvents, more preferably DMF to improve coupling. Coupling was repeated when Kaiser test showed positive. Unreacted sites on the growing chain were capped using 9.4 ml of acetic anhydride and 16.5 ml of NMM in DMF (5 Eq, 9.97 mmol:5 Eq, 15 mmol).

The final step involves the deblocking of the Fmoc group of Fmoc-D-Cys (Trt)-OH and acetylation of the amino terminal using 9.4 ml of acetic anhydride and 16.5 ml of NMM as a Base (5 Eq, 9.97 mmol: 7.5 Eq, 15 mmol). The heptamer bound to peptidyl resin is washed with 318 ml of Dichloromethane for 2 min each for 3 cycles and dried under vacuum.
Weight of peptide obtained 68.15 g

### EXAMPLE 2: Cleavage of the Acetylated Heptamer of Etelcalcetide from the Peptidyl Resin

Two different approaches were used for cleaving cleavage cocktails are described below:

### A) Cleavage Mixture of TFA: TIS: DMS

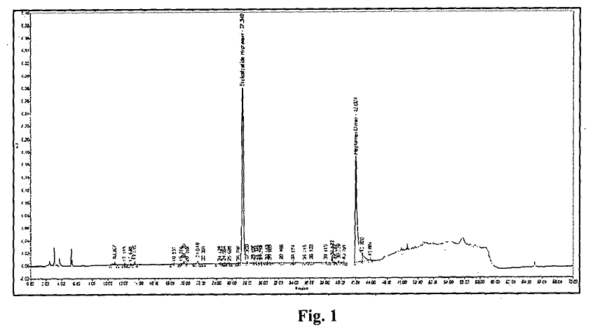

The dried heptamer bound to peptidyl resin obtained from Example 1 was cleaved using a cleavage mixture consisting of TFA: TIS: DMS. Ratio of the solvents used in cocktail ranged for TFA (80 to 95), TIS (2.5 to 10) and DMS (2.5 to 10) (%v/v). Preferred ratio of the solvents used is 90:6.5: 3.5 (%v/v). Concentration of cleavage cocktail ranged from 10-30 ml/g of peptidyl resin; preferably 20 ml/g for 3 to 4 Hrs. 1.36 L of the cocktail is prepared by mixing 1.2 L of TFA, 88.4 ml of TIS and 47.6 ml of DMS. 68.15 g of peptidyl resin is added to the cocktail. Reaction is stirrcd for 4 hours and filtered. The filtrate was concentrated and precipitated in 10 volumes of cold DIPE/MTBE. The precipitate obtained was filtered and washed with DIPE/MTBE and dried under vacuum to obtain the crude peptide. Analytical chromatogram of the crude peptide obtained in this step is shown in the FIG. 1.
Crude Purity: 50% Heptamer Etelcalcetide and 40% Dimer Etelcalcetide
Yield: 18 g
% Yield: 96.7%

### B) Cleavage Mixture of TFA: TIS : DMS: TCEP: Water

The dried peptidyl resin was cleaved using a cleavage mixture comprising of TFA: TIS: DMS: TCEP: Water in the ratio TFA (80 to 95) TIS (2 to 10) DMS (2 to 10) TCEP (1 to 5), Water (0.5 to 5) (%v/v). Preferred ratio of the solvents used is 85: 6.5: 3.5:1.5:1 at a concentration of 10-30 ml/g of peptidyl resin; preferably 20 ml/g for 3 to 4 Hrs.

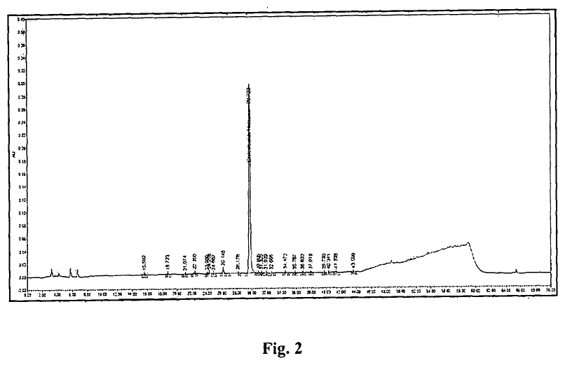

1.36 L of the cocktail was prepared by mixing 1.15 L of TFA, 88.4 ml of TIS and 47.6 ml of DMS., 20.4 g of TCEP and 13.6 ml of Water, 68.15 g of peptidyl resin was added to the precold cocktail. Reaction was stirred for 4 hours and filtered. The filtrate was concentrated and precipitated in 10 volumes of cold DIPE/MTBE. The precipitate obtained was filtered and washed with DIPE/MTBE and dried under vacuum to obtain the crude peptide. Analytical chromatogram of the crude peptide obtained in this step is shown in the FIG. 2.
Crude peptide Purity obtained: 92.5%
Yield: 18.4 g
% Yield: 98.3%

### Example 3: Reduction of the Crude Peptide Before Purification

Crude peptide obtained in Example 2 A, 7 g (7.53 mmol) is dissolved in a 350 ml of aqueous buffer at pH 2.5, preferably 0.1-2% Perchloric acid in water. pH was adjusted to 2.5 with Sodium hydroxide. Concentration of peptide can be used in range from 5 mg/ml to 50 mg/ml more preferably 20 mg/ml.

1.3 g (4.52 mmol) of TCEP.HCl is added to the mixture and the pH of the solution was adjusted to 3 to 5.5 preferably 4.5 using TEA/Sodium hydroxide/Ammonia. The reaction is stirred for 35 min to 180 min preferably 75 min either at 10° C. to ambient temperature (RT), pH adjusted to 2.5 to 3.5 using Hydrochloric acid/ Perchloric acid/ Acetic acid/ TFA preferably Perchloric acid.

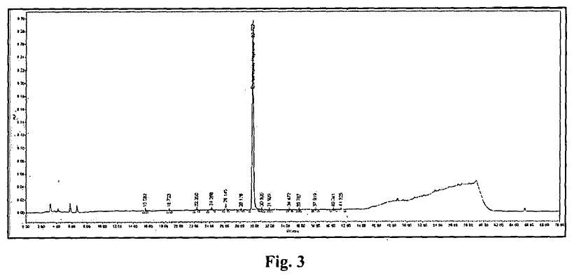

Analytical chromatogram of the crude peptide obtained in this step is shown in the FIG. 3.
Purity of Heptamer Etelcalcetide: 93%

### Example 4: Purification of Heptamer Etelcalcetide by HPLC

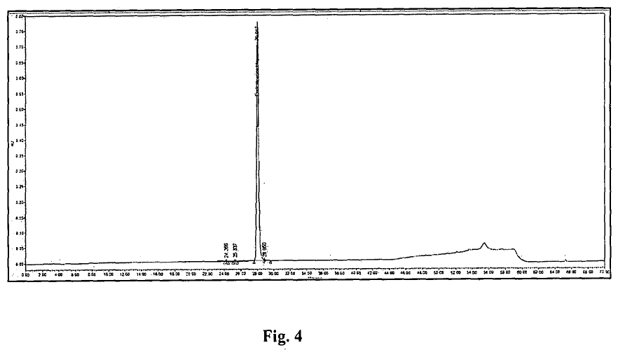

The crude peptide generated in Example 3 is purified using a Reverse phase HPLC column consisting of C 18, 10 micron 100 A silica. The mobile phase comprised of a buffer of 0.1-3% v/v Perchloric acid pH 2.5 with Sodium hydroxide in water and Acetonitrile. The peptide was purified using a gradient composition of Acetonitrile with the mobile phase A mentioned in the table below. The collected fraction are analyzed by an Analytical HPLC. Analytical chromatogram of the purified peptide obtained in this step is shown in the FIG. 4.
Purity of heptamer Etelcalcetide fraction: 99.8%
Yield: 3.0 g
% Yield: 50%

| | |
|---|---|
| Instrument | Preparative HPLC |
| Mobile Phase A | 1% Perchloric Acid pH 2.5 with Sodium hydroxide |
| HPLC media | C18, 10 micron, 100 A Silica |
| Column Dimension | 76 mm × 250 mm |
| Flow rate | 138 ml/min |
| Detector wavelength | 220 nm |

### Example 5: Oxidation of Heptamer Etelcalcetide to Etelcalcetide and Purification

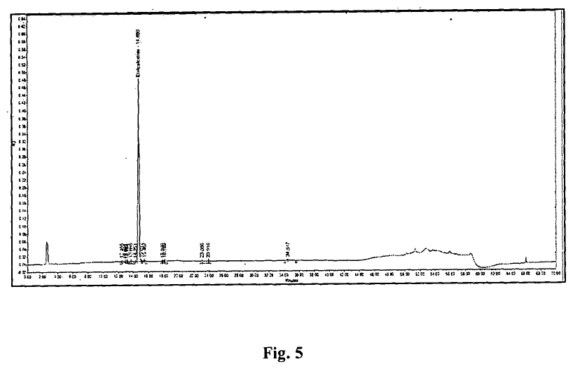

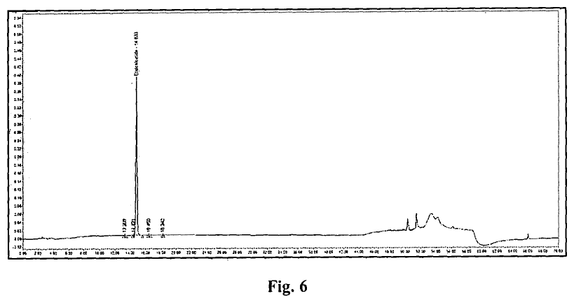

3.85 g of the purified heptamer Etelcalcetide (4.14 mmol) obtained in Example 4 is diluted to obtain a concentration of 0.8-2.0 mg /ml preferably 1.0 mg/ml. Oxidation is carried out by adding 10.1 g (62.15 mmol) of Cysteine hydrochloride monohydrate and adjusting the pH of the mixture to 7 to 8.5 preferably 8.0 using sodium hydroxide solution. 8.0 ml (93.23 mmol) of 35% Hydrogen peroxide is added to the reaction and the reaction is allowed to continue for 15 mins. After the completion of time the reaction pH is acidified to pH 3.0 with perchloric acid and filtered. Analytical chromatogram of the peptide obtained after oxidation reaction in this step is shown in the FIG. 5. The filtered solution is loaded onto HPLC and purified. The HPLC column comprises C 18, 10 micron silica. The mobile phase B comprised of a buffer of 0.05-1.0 M Ammonium Chloride pH 3.0 with HCl. Methanol or Ethanol or Acetonitrile is used as the eluting solvent in the gradient mode to purify the peptide. The collected fractions are analyzed by an Analytical HPLC. Analytical chromatogram of the purified peptide obtained in this step is shown in the FIG. 6.
Purity of Collected fraction: 99.8%
Yield: 3.0 g
% Yield: 43%

| | |
|---|---|
| Instrument | Semi Preparative HPLC |
| Mobile Phase B | 0.1M NH₄Cl pH 3.0 with HCl and Methanol |
| HPLC media | C18, 10 micron, 100A Silica |
| Column Dimension | 76 mm × 250 mm |
| Flow rate | 80 ml/min |
| Detector wavelength | 220 nm |

### Example 6: Isolation and Lyophilization of Etelcalcetide as a Chloride Salt

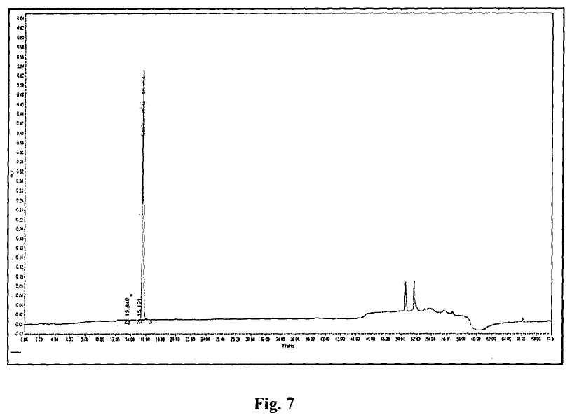

Purified Etelcalcetide 3.0 g in eluting buffer obtained from Example 5 was concentrated under reduced pressure and isolated by precipitating it in 1.5 litre alcoholic solvent or with composition of mixture of solvents such as methanol, ethanol, isopropyl alcohol with either acetonitrile, ethyl acetate, acetone. The precipitate is isolated by centrifugation or by filtration. The precipitate is washed with the above solvent mixture and centrifuged to obtain the wet cake. The wet cake obtained is again washed with an acidified solution of acetone or a mixture of acetone and ethanol, which consists of 0.05% to 2% hydrochloric acid in acetone, or mixture of acetone: ethanol, most preferably 0.25% hydrochloric acid in acetone. The precipitate is again centrifuged to obtain a wet cake. The wet cake obtained is dissolved in water and concentrated to 50-300 mg/ml. The concentrated sample is lyophilised to obtain end product as highly purified Etelcalcetide hydrochloride. The lyophilized amorphous product has bulk density of 0.6 to 0.8 g/cm3. Analytical chromatogram of the purified peptide obtained in this step is shown in the FIG. 7.
Purity: 99.8%
Yield: 3.0 g

### Example 7: Isolation and Lyophlization of Etelcalcetide as a Chloride Salt

Purified Etelcalcetide 3.0 g in eluting buffer obtained from Example 5 was concentrated under reduced pressure and isolated by precipitating it in 1.5 liter alcoholic solvent or with composition of mixture of solvents such as methanol, ethanol, isopropyl alcohol with either acetonitrile, ethyl acetate, acetone. The precipitate is isolated by centrifugation or by filtration. The precipitate is washed with the above solvent mixture and centrifuged to obtain the wet cake. The wet cake obtained is dissolved in water and concentrated to 50-300 mg per ml. The concentrated sample at 200mg/ml is mixed with a chilled dilute solution of Hydrochloric acid in water. The Hydrochloric acid is added to water maintained at 2-8° C. at a concentration of 2% to 6% with respect to the peptide. The solution is lyophilised to obtain end product as highly purified Etelcalcetide hydrochloride. The lyophilized amorphous product has bulk density of 0.6 to 0.8 g/cm³. Analytical chromatogram of the purified peptide obtained in this step is shown in the FIG. 7.
Purity: 99.8%
Yield: 3.0 g

1. An improved process for synthesis of Etelcalcetide or salt or precursor thereof as set forth in Formula I by an orthogonal Fmoc strategy comprising of: 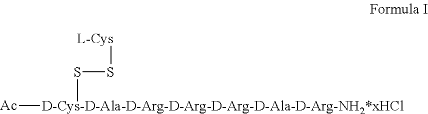
i. covalently linking a Fmoc-D-Arg(Pbf)-OH to polystyrene based solid resin support,
ii. removing the α-NH2 protecting group from Fmoc-D-Arg(Pbf)-solid support to obtain a free α-NH2 group,
iii. coupling the second Fmoc-D-Ala to the D-Arg(Pbf)-solid support, by activating the amino acid by DIC/HOBT in the presence of organic solvent,
iv. deprotecting the Fmoc group by a deprotectant,
v. repeating steps ii), iii), iv) for assembling the heptapeptide H-D-Cys(Trt)-D-Ala-D-Arg(Pbf)-D-Arg(Pbf)-D-Arg(Pbf)-D-Ala-D-Arg(Pbf)-solid resin support,
vi. acetylating the N-terminal group to produce acetylated heptapeptide Ac-D-Cys(Trt)-D-Ala-D-Arg(Pbf)-D-Arg(Pbf)-D-Arg(Pbf)-D-Ala-D-Arg(Pbf)-solid resin support,
vii. cleaving the heptapeptide from solid resin support using cleavage cocktail consisting of TFA in the range of 80 to 95% V/V, TIS in the range of 2 to 10% V/V, DMS in the range of 2 to 10% V/V, TCEP in the range of 1 to 5 V/V, water in the range of 1 to 5% V/V, preferably TFA: TIS: DMS: TCEP: water in the ratio of 85: 6.5: 3.5:1.5:1 (%v/v); to obtain crude heptamer-etelcalcetide as set in the formula II of at least 90% peptide purity, 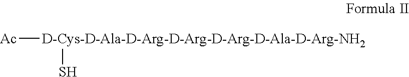
viii. optionally purifying the crude heptamer-etelcalcetide as set in the formula II of step vii) by chromatography wherein said peptide has a purity of ≥98%,
ix. oxidizing heptamer-etelcalcetide as set in the formula II of step viii) with free single cysteine in presence of hydrogen peroxide to obtain Etelcalcetide as set forth formula I
x. purifying etelcalcetide hydrochloride of step ix) by reverse phase HPLC to a purity of at least 99.8%
xi. precipitating, adjusting chloride salt content and lyophilizing the concentrated purified peptide solution of step x).

2. (canceled)

3. The improved process for synthesis of Etelcalcetide or salt or precursor thereof as claimed in claim 1, wherein cleavage of heptapeptide from the solid resin support is carried out using mixture of solvents selected from a group consisting of TFA, TCEP, TIS, Water, DTT, DMS, DODT.

4. The improved process for synthesis of Etelcalcetide or salt or precursor thereof as claimed in claim 1, wherein cleavage of heptapeptide from the solid resin support is done wherein concentration of peptidyl resin is 10-30 ml/g, preferably 20 ml/g.

5. The improved process for synthesis of Etelcalcetide or salt or precursor thereof as claimed in claim 1, wherein the purification of crude heptamer-etelcalcetide to a purity of ≥99% carried by chromatography is by RP-HPLC by isocratic and/or gradient mode.

6. The improved process for synthesis of Etelcalcetide or salt or precursor thereof as claimed in claim 1, wherein the eluent for RP-HPLC purification of crude heptamer-etelcalcetide by gradient mode comprises of perchloric acid buffer system with pH in the range of 3 to 5, preferably 2.5 as an aqueous phase and acetonitrile as an organic phase with isolated yield of at least 50% and purity of at least 99%.

7. The improved process for synthesis of Etelcalcetide or salt or precursor thereof as claimed in claim 1, wherein the purification of etelcalcetide and its conversion to its hydrochloride salt with purity of ≥99.80% by chromatography is by RP-HPLC by isocratic and/or gradient mode.

8. The improved process for synthesis of Etelcalcetide or salt or precursor thereof as claimed in claim 1, wherein the eluent for RP-HPLC purification of Etelcalcetide by gradient mode comprises of Ammonium chloride in Hydrochloric acid buffer system as an aqueous phase with pH in the range of 2 to 5, preferably 3 and an organic phase comprising from solvents selected from methanol, ethanol or acetonitrile preferably methanol with isolated yield of at least 43% and purity of at least 99.8%.

9. The improved process for synthesis of Etelcalcetide or salt or precursor thereof as claimed in claim 1, wherein the precipitation of purified Etelcalcetide hydrochloride is by solvent system comprising of mixture of solvents consisting from group of methanol, ethanol, isopropyl alcohol, acetonitrile, ethyl acetate, acetone either alone or in combination thereof, most preferably combination of ethanol and acetone.

10. The improved process for synthesis of Etelcalcetide or salt or precursor thereof as claimed in claim 1, wherein the precipitation of purified Etelcalcetide hydrochloride is followed by washing the precipitate obtained with an acidified solution of acetone or a mixture of acetone and ethanol, which consists of 0.05% to 2% hydrochloric acid in acetone, or mixture of acetone: ethanol, most preferably 0.25% hydrochloric acid in acetone to ensure the counter ion content of the Etelcalcetide is maintained within a narrow range of 4 to 5 equivalents with respect to the peptide.

11. The improved process for synthesis of Etelcalcetide or salt or precursor thereof as claimed in claim 1, wherein the adjustment of chloride salt content involves addition of chilled solution of dil. Hydrochloric acid in water to the peptide, the concentration of which is maintained at concentration of 50 to 200 mg/ml more preferably 200 mg/ml.

12. The improved process for synthesis of Etelcalcetide or salt or precursor thereof as claimed in claim 1, wherein the lyophilization of the purified Etelcalcetide hydrochloride is done at the high peptide concentration ranging from 50 to 300 mg/ml, more preferably at 200 mg/ml to obtain an amorphous peptide with a high bulk density of 0.6-1.0 g/cm³.

13. An improved process for synthesis of Etelcalcetide or salt/precursor thereof as set forth in Formula 1 by an orthogonal Fmoc strategy comprising of:
i. covalently linking a Fmoc-D-Arg(Pbf)-OH to polystyrene based solid support,
ii. removing the α-NH2 protecting group from Fmoc-D-Arg(Pbf)-solid resin support to obtain a free α-NH2 group,
iii. coupling the Fmoc-D-Ala to the D-Arg(Pbf)-solid resin support, by activating the amino acid by DIC/HOBT in the presence of organic solvent,
iv. deprotecting the Fmoc group by deprotectant,
v. repeating steps ii), iii), iv) for assembling the heptapeptide H-D-Cys(Trt)-D-Ala-D-Arg(Pbf)-D-Arg(Pbf)-D-Arg(Pbf)-D-Ala-D-Arg(Pbf)-solid resin support,
vi. acetylating the N-terminal group to produce acetylated heptapeptide Ac-D-Cys(Trt)-D-Ala-D-Arg(Pbf)-D-Arg(Pbf)-D-Arg(Pbf)-D-Ala-D-Arg(Pbf)-solid resin support,
vii. cleaving the heptapeptide from solid resin support using cleavage cocktail consisting of TFA in the range of 80 to 95% V/V, TIS in the range of 2.5 to 10% V/V, DMS in the range of 2.5 to 10% V/V, preferably TFA: TIS: DMS in the ratio of 90: 6.5: 3.5 (% v/v) to obtain mixture of crude heptamer-etelcalcetide as set in the formula II and dimer-etelcalcetide as set in the formula III, 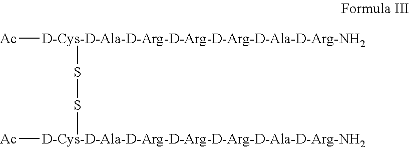
viii. pretreating the crude mixture of heptamer-etelcalcetide and dimer- etelcalcetide of step vii) with Tris(2-carboxyethyl)phosphine hydrochloride in presence of perchloric acid buffer having concentration ranging from 0.1 to 2% preferably 1% and pH ranging from 2 to 5, preferably 2.5 to obtain heptamer-etelcalcetide of at least 90% peptide purity,
ix. purifying the crude heptamer-etelcalcetide as set in the formula II of step viii) by chromatography wherein said peptide has a purity of ≥98%,
x. oxidizing heptamer-etelcalcetide as set in the formula II of step ix) with free single cysteine in presence of Hydrogen peroxide to obtain Etelcalcetide as set forth formula I
xi. purifying Etelcalcetide hydrochloride of step x) by reverse phase HPLC to a purity of at least 99.8%
xii. precipitating, adjusting chloride salt content and lyophilizing the concentrated purified peptide solution of step xi).

14. The improved process for synthesis of Etelcalcetide or salt or precursor thereof as claimed in claim 13, wherein cleavage of heptapeptide from the solid resin support is carried out using mixture of solvents selected from a group consisting of TFA, TCEP, TIS, Water, DTT, DMS, DODT.

15. The improved process for synthesis of Etelcalcetide or salt or precursor thereof as claimed in claim 13, wherein cleavage of heptapeptide from the solid resin support is done wherein concentration of peptidyl resin is10-30 ml/g, preferably 20 ml/g.

16. The improved process for synthesis of Etelcalcetide or salt or precursor thereof as claimed in claim 13, wherein the purification of crude heptamer-etelcalcetide to a purity of ≥99% carried by chromatography is by RP-HPLC by isocratic and/or gradient mode.

17. The improved process for synthesis of Etelcalcetide or salt or precursor thereof as claimed in claim 13, wherein the eluent for RP-HPLC purification of crude heptamer-etelcalcetide by gradient mode comprises of perchloric acid buffer system with pH in the range of 3 to 5, preferably 2.5 as an aqueous phase and acetonitrile as an organic phase with isolated yield of at least 50% and purity of at least 99%.

18. The improved process for synthesis of Etelcalcetide or salt or precursor thereof as claimed in claim 13, wherein the purification of etelcalcetide and its conversion to its hydrochloride salt with purity of ≥99.80% by chromatography is by RP-HPLC by isocratic and/or gradient mode.

19. The improved process for synthesis of Etelcalcetide or salt or precursor thereof as claimed in claim 13, wherein the eluent for RP-HPLC purification of Etelcalcetide by gradient mode comprises of Ammonium chloride in Hydrochloric acid buffer system as an aqueous phase with pH in the range of 2 to 5, preferably 3 and an organic phase comprising from solvents selected from methanol, ethanol or acetonitrile preferably methanol with isolated yield of at least 43% and purity of at least 99.8%.

20. The improved process for synthesis of Etelcalcetide or salt or precursor thereof as claimed in claim 13, wherein the precipitation of purified Etelcalcetide hydrochloride is by solvent system comprising of mixture of solvents consisting from group of methanol, ethanol, isopropyl alcohol, acetonitrile, ethyl acetate, acetone either alone or in combination thereof, most preferably combination of ethanol and acetone.

21. The improved process for synthesis of Etelcalcetide or salt or precursor thereof as claimed in claim 13, wherein the precipitation of purified Etelcalcetide hydrochloride is followed by washing the precipitate obtained with an acidified solution of acetone or a mixture of acetone and ethanol, which consists of 0.05% to 2% hydrochloric acid in acetone, or mixture of acetone:
ethanol, most preferably 0.25% hydrochloric acid in acetone to ensure the counter ion content of the Etelcalcetide is maintained within a narrow range of 4 to 5 equivalents with respect to the peptide.

22. The improved process for synthesis of Etelcalcetide or salt or precursor thereof as claimed in claim 13, wherein the adjustment of chloride salt content involves addition of chilled solution of dil. Hydrochloric acid in water to the peptide, the concentration of which is maintained at concentration of 50 to 200 mg/ml more preferably 200 mg/ml.

23. The improved process for synthesis of Etelcalcetide or salt or precursor thereof as claimed in claim 13, wherein the lyophilization of the purified Etelcalcetide hydrochloride is done at the high peptide concentration ranging from 50 to 300 mg/ml, more preferably at 200 mg/ml to obtain an amorphous peptide with a high bulk density of 0.6-1.0 g/cm³.
